(19) United States
(12) Patent Application Publication
(10) Pub. No.: US 2020/0289443 A1
(43) Pub. Date: Sep. 17, 2020

(54) Method of Citric Acid as a Biochemical Enhancer

(71) Applicant: Vivi Robyn Stafford, Los Angeles, CA (US)

(72) Inventor: Vivi Robyn Stafford, Los Angeles, CA (US)

(21) Appl. No.: 16/299,159

(22) Filed: Mar. 12, 2019

(51) Int. Cl. A61K 31/194; A61K 9/00; A61K 38/48

(52) U.S. Cl. CPC A61K 31/194; A61K 38/4893; A61K 9/0053; A61K 9/0019

(57) ABSTRACT

Methods for the metabolic manipulation with citric acid prolonging and amplifying the effects of pharmacological therapies including neurotoxins and citrate through taxis and its effects on acetylcholine, immunological factors, calcium and the localized depletion of o2. Citric acid provides for longer lasting medical grade neurotoxin results related to paralysis by its effects, dose related, on acetylcholine and through commutative irritating responses in tissues. Outcomes are through acetylcholine amplification and immunoreactivity, and a lack thereof, providing longer lasting results through acetylcholine biochemical taxis. Pertinent to these effects, neurotoxins such as botulinum toxin paralyze human muscle tissue which is enhanced by citric acid through actions on acetylcholine, meanwhile, there is a localized irritating factor derived from the citric acid increasing a cellular response similar to the responses in wound healing. Citric acid, an irritant, does not provoke an immune response. The sequestering of calcium by the citric acid assists the botulinum toxin minimizing the influx of calcium related to acetylcholine. In addition, this phenomenon provides for overwhelming the nicotinic receptors with acetylcholine through shunting which enhances the patient's ability to have a more stable mood, increased pleasurable experiences, decreased pain and can improve the cognition of those with diseases related to the nicotinic acetylcholine receptors. Citric acid induced shunting of acetylcholine to the nicotine receptors can mitigate addiction cravings related to eating, smoking and opioids. In addition, the mutated immune response by the citric acid provides for more effective treatments to those with immunity to neurotoxin therapy.

### STATEMENT REGARDING FEDERALLY SPONSORED RESEARCH

This invention was not made with U.S. government support.

### BACKGROUND

Botulinum toxin is produced from the bacteria Clostridium botulinum. The botulinum toxin excretes exotoxins which are considered deadly. The Clostridium botulinum exotoxins include A, B, C1, C2, D, E, F, and G. The toxins produced by Clostridium botulinum block the release of acetylcholine. The toxin has a high affinity for the presynaptic surface of cholinergic neurons where it binds irreversibly and selectively to the receptors. These nerve terminals are permanently blocked and function is recovered with sprouting of nerve terminals and formation of new synaptic contacts; however, the concomitant presence of exogenous citric acid, the irritating factors along with its zero ability to provoke an immune response and calcium interference delays the response to sprouting new nerve terminals and the formation of new synaptic contacts. Currently, without citric acid used as a supportive agent, Botulinum toxin for commercial use has an anticipated term of only three months. The result of the botulinum toxin at rest with the simultaneous use of citric acid extends results beyond three years. The concomitant injections of solutions of citric acid and neurotoxins simultaneously, including as mixed solutions, provides for exceedingly long lasting and long term results to the extent of providing greater longevity in outcomes. The combination injection of neurotoxins such as Clostridium Botulinum and Citric Acid maximizes, prolongs, amplifies and magnifies the paralytic effects of the Neurotoxin. These actions are induced through the manipulation of acetylcholine through the use of citric acid which affects the nerve action potential related to botulinum toxin. The acetylcholine is potentiated within the neuronal synapse in response to the citric acid which potentiates the effects. The neurotoxin potentiation variably being through a form of inhibition of acetylcholinesterase, of inhibition of the uptake of neurotransmitter, of an increase of acetylcholine in the presynaptic plasma membrane, of the inhibition of the release of neurotransmitter, of the shunting of acetylcholine to other receptors including nicotinic receptors, of the sequestering of calcium by the citrate, of the effects of o2 in the tissues in relations to the augmenting of the Krebs cycle, of the inhibition of the efficient transportation of choline for the purposes of providing recycled acetylcholine for muscle contractions and of the increase in acetylcholine availability followed by a decrease in acetylcholine availability. There is an ultimate decrease in the availability of acetylcholine potentiating the binding effects or the effects of binding of the neurotoxin on the target plasma membrane; notwithstanding, the effects related to calcium sequestration, o2 taxis and immune factor responses.

The irritating effects of citric acid are more profound in a relaxed tissue in an environment where muscle contractions provide for assisting in localized circulation. Presynaptic terminals of cholinergic nerves are susceptible to the botulinum toxin, notwithstanding these terminals are considered labile in relations to botulinum toxin and the mitigation of the reuptake of choline into the presynaptic terminal for recycling amplifies the paralysis related to neurotoxins, specifically botulinum toxin A, B, C1, C2, D, E, F, G and the like. Citric acid binds calcium which assists in the neurotoxins being more efficient in blocking the calcium dependent channels which release acetylcholine for muscle contractions. Additionally, toxins produced by Clostridium botulinum block inhibitory and excitatory postsynaptic action. As a result, antibodies theoretically are less abundant due to a decrease in local circulation related to muscle contractions where quelling the immune response to a neurotoxin makes the neurotoxin more potent in terms of effectiveness in the environment of administered citric acid.

Acetylcholine is a compound which occurs throughout the nervous system of humans. Acetylcholine is a neurotransmitter which functions in the neuromuscular junction. It is an organic substance where its release is required to activate muscles. Acetylcholine is an ester of acetic acid and choline. Substances that affect acetylcholine through inhibition are considered anticholinergics. Acetylcholine is the substance involved with muscle contractions where the inhibition of acetylcholine causes paralysis and the increase secretion of it causes contractions; therefore, anticholinergics can cause paralysis, convulsions and also death. Acetylcholine does not pass lipid membranes. Acetylcholine is synthesized in certain neurons by the enzyme choline acetyltransferase from the compounds choline and Acetyl-CoA. Neurotoxins can work by inhibiting acetylcholinesterase, thus leading to excess acetylcholine at the neuromuscular junction as the breakdown of acetylcholine in inhibited. The rate limiting step in the synthesis of acetylcholine is through the high affinity choline uptake. This is a highly regulated process and this process provides for the majority of the choline uptake in the nerve terminal. And it is this rate limiting step in which an abundance of choline can essentially bottle neck in the post synaptic terminal causing the excess acetylcholine and its precursors through feedback to be shunted elsewhere, giving the neurotoxins a greater affinity for binding its target.

The citric acid cycle occurs in the mitochondria. The citric acid cycle generates energy through the oxidation of acetate. Neurons metabolize choline into acetylcholine through an additional acetyl group donated by acetyl Co-A. The neuronal metabolized acetylcholine is exported out of the neuron via a vesicle. Reaction mechanism includes the steps for a reactant to become a product. In the case of acetylcholine, it is produced through the esterification of acetic acid and choline. Reaction intermediates are formed in one step and then consumed later in a reaction mechanism step. The slowest step in the mechanism is the rate limiting step. The rate limiting step in acetylcholine synthesis is once the acetylcholine's job is done in the synapse. Synaptic acetylcholinesterase breaks the acetylcholine back down into acetate anions and choline. In the Krebs cycle Acetyl CoA and oxaloacetate are induced into a condensation reaction by citrate synthase producing citric acid. An increase in the concentration in the cellular environment of citric acid would allow the Krebs cycle to produce energy through the process of aerobic respiration. As a result, the Acetyl CoA would be more metabolically available for the production of acetylcholine with the application of exogenous citric acid. Additionally, the citric acid administered upregulates the Krebs cycle which continues in an aerobic environment. Meanwhile, the botulinum toxin is a product of an anaerobic environment providing an activated Krebs cycle environment ideal for increasing the effectiveness of the toxin on the cell membrane target due to oxygen depletion through the citric acid induced over utilization of aerobic respiration. Choline reuptake is the rate limiting step in synthesis of acetylcholine. Once acetylcholine is no longer needed within a neuronal synapse, it is broken down by synaptic acetylcholinesterase into acetate ions and choline. Acetylcholinesterase terminates synaptic transmission. As a result, the recycling of acetylcholine can be minimized through a decrease of acetylcholinesterase. Neurotransmitter uptake also inhibits the signaling of neurotransmitters whereas the increased production of acetylcholine would provide negative feedback for the production of additional neurotransmitter enhancing the effects of botulinum toxin. Evidence has shown that Botulinum Toxin does not significantly reduce the voltage activated calcium current in the nerve endings. However, calcium binding is a rate limiting step in the exocytosis of the neurotransmitter acetylcholine where again there can be bottle necking in the process potentiating the effects of botulinum toxin. Citric acid is a chelator of calcium to the extent that an environment can be effectively depleted of calcium due to the chelation mechanism of citric acid. The binding of calcium decreases availability of calcium for muscle contraction.

In the Krebs Cycle, exogenous Citric acid increases the productivity of the cycle which provides an ample supply of acetic acid for the production of acetylcholine. After acetylcholine triggers a muscular contraction, the action is chemically inhibited through the metabolism of acetylcholine to choline. Citric acid injected with the Neurotoxin potentiates its effects as the citric acid allows the Krebs cycle mechanism to provide energy; meanwhile, not utilizing the Acetyl CoA in the Krebs reaction mechanism. This allows the acetyl CoA to be more readily available for the synthesis of acetylcholine; therefore, acetylcholine's increase in productivity provides for a negative feedback reaction, more efficient binding of the neurotoxin to acetylcholine receptors and the shunting of acetylcholine to other receptors. The citric acid cycle being propagated forward through an increase in concentration of citric acid, means there would be more acetyl coA available for metabolizing. As a result, more acetyl coenzyme A is available for utilization where it is metabolized into Acetylcholine meaning the neurotoxin cosmetic results would be more profound. The profound effects of the citric acid dumping phenomenon allows the neurotoxins to be more efficient in their metabolic actions including increased binding to receptors and longer lasting integrity of the neurotoxins action meaning a delay in the regeneration of new nerve terminals. In addition, the increase of the presence of citric acid means that calcium chelation would occur which provides a more stable plasma membrane for the neurotoxin to attach and to be brought into the cell via endocytosis.

### BRIEF DESCRIPTION OF THE DRAWINGS

The patent of application file contains at least one drawing executed in color. Copies of this patent or patent application publication with color drawings will be provided by the office upon request and payment of the necessary fee.

The present invention is illustrated by way of example in the accompanying drawings. The drawings should be understood as illustrative rather than limiting.

FIG. 1 illustrates condition of a patient before and after treatment using an embodiment of the invention.

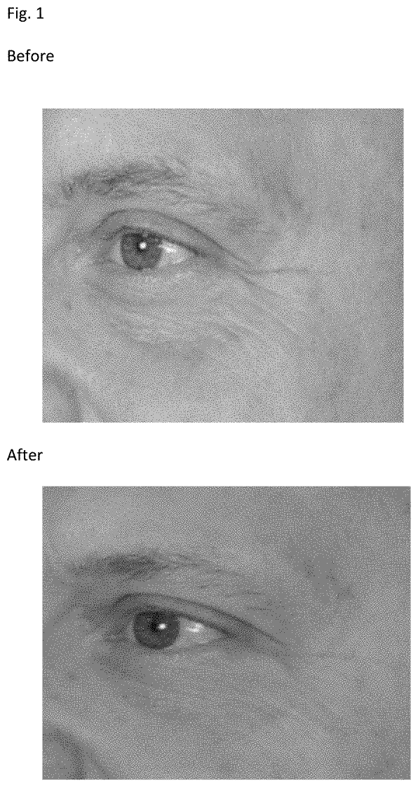

FIG. 2 illustrates condition of another patient before and after treatment using an embodiment of the invention.

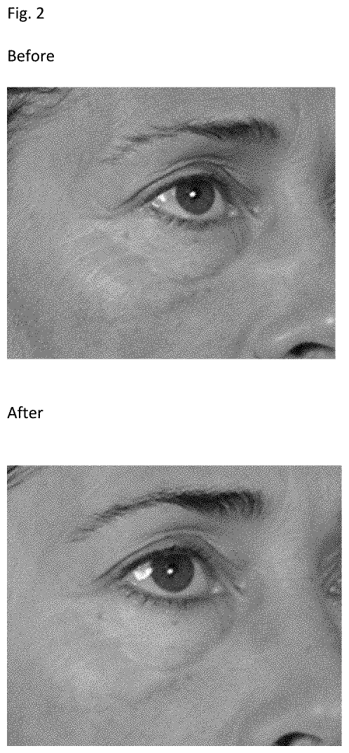

FIG. 3 illustrates condition of yet another patient before and after treatment using an embodiment of the invention.

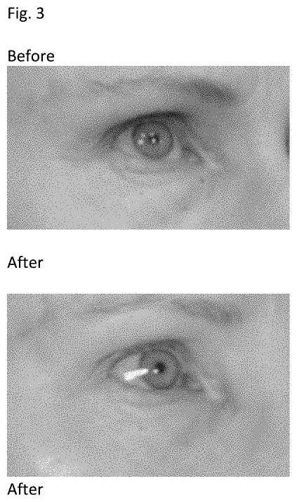

FIG. 4 illustrates condition of another patient before and after treatment using an embodiment of the invention.

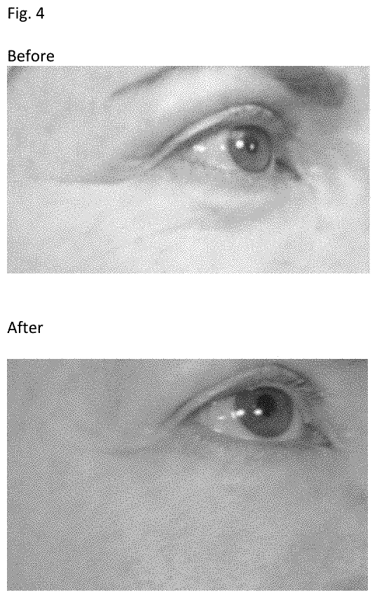

### DETAILED DESCRIPTION

A Method of Citric Acid as a Biochemical Enhancer is provided. The specific embodiments described in this document represent example instances of the present invention, and are illustrative in nature rather than restrictive.

### Brief Description of the Invention:

Citric acid is found in the cells of humans but can be manufactured and processed. Neurotoxins are always foreign to an individual and are introduced either medically or through an exposure in the environment. Treatments with neurotoxins in the healthcare environment are fraught with uncertainty. For instance, the treatments are not markedly long lasting, a larger dose may be required for a desired effect adding to the concern that there is risk of developing a level of immunity to the treatments. The recipient frequently believes the treatment has toxicity levels and frequently requests lesser doses than is required for their desired effect. Citrate mixed with the botulinum toxin effects the binding of botulinum toxin amplifying the results with zero risks of there being any additional complications related to immune responses. And the paralysis with the concurrent irritant from the citrate provides for permanent botulinum toxin results at rest related to it stimulating fibroblast activity in an environment at rest due to paralysis for a period of time to reversing signs of aging. Additionally, the method of citric acid shunting excess acetylcholine to nicotinic receptors means there is more ability for the body to utilize dopamine which enhances moods, can treat dopamine resistant disorders and also can decrease cravings in addictions such as obesity, opioid dependence and treats pain.

### Specification:

Diluted botulinum toxin is placed into solution through dilution in the standard pharmaceutical fashion. The standardization of the dose of botulism for commercial use is one unit of botulism toxin corresponds to the lethal median intraperitoneal dose does in female Swiss-Wester mice (LD50) with serotype A. This standardized pharmaceutical fashion is the generalized inactive single polypeptide chains with a molecular mass of about 150 kD with a high degree of amino acid sequence. Normal saline in the amount of 0.5 cc is drawn and mixed with sodium citrate 0.25 cc and dextrose 10 with a buffered sodium citrate added solution of 0.109 ml 3.2% Sodium citrate buffered. The remaining solution of citric acid is combined with botulinum toxin with ½ a unit applied to the calculated dose compared to 1 unit of botulinum toxin when augmented with citric acid. The crow's feet are injected with citrate solutions as a drug enhancing agent just deep to the crow's feet, and into the glabellum and the forehead and to any other area that is to be treated with the combined solution. The injections are placed into the belly of the muscles with bolus style injections. For a more enhanced result after, the injection of the citric acid, patient takes the citric acid orally for 13 days two to three times daily. For an enhanced result a patient can have an injection independently or oral citric acid independently.

One skilled in the art will appreciate that although specific examples and embodiments of the system and methods have been described for purposes of illustration, various modifications can be made without deviating from present invention. Moreover, features of one embodiment may be incorporated into other embodiments, even where those features are not described together in a single embodiment within the present document.

1. A method for administering citric acid in humans, or salts thereof, as an active drug by its biochemical enhancing effects on the performance of pharmaceutical drugs by improving longevity, improving amplification and improving performance through, one or a combination of, it manipulating acetylcholine or choline, it causing localized irritation, it inhibiting immunological factors, it augmenting immunological factors, it chelating or sequestering calcium and it effecting o2 gradients.

2. The method of claim 1, wherein said administration is through injection, oral administration or ingestion.

3. The method of claim 1, wherein said administration is as a mixture with the pharmaceutical it is enhancing.

4. The method of claim 1, wherein said administration is given independently as a pharmaceutical enhancer before, after or during the treatment of the pharmaceutical it is enhancing.

5. The method of claim 1, wherein said injection of citric acid, or salts thereof, providing for drug enhancement through acetylcholine manipulation enhancing the longevity of the paralysis related to commercial neurotoxins.

6. The method of claim 1, wherein said oral administration of citric acid, or salts thereof, providing for drug enhancement through acetylcholine manipulation enhancing the longevity of the paralysis to commercial neurotoxins.

7. The method of claim 1, wherein the injection of citric acid, or salts thereof, providing for drug enhancement in a local environment of irritation enhancing the appearance of commercial neurotoxins at rest.

8. The method of claim 1, wherein the oral administration of citric acid, or salts thereof, providing for drug enhancement for a local environment of irritation enhancing the appearance of commercial neurotoxins at rest.

9. The method of claim 1, wherein the injection of citric acid, or salts thereof, providing for drug enhancement for a local environment of irritation enhancing the longevity beyond a year in the appearance of commercial neurotoxins at rest.

10. The method of claim 1, wherein the oral administration of citric acid, or salts thereof, providing for drug enhancement for a local environment of irritation enhancing the longevity beyond a year in the appearance of commercial neurotoxins at rest.

11. The method of claim 1, wherein the injection of citric acid, or salts thereof, providing for drug enhancement and prolonging the paralytic effects of commercial neurotoxins for durations longer than commercial neurotoxin use without the enhancer.

12. The method of claim 1, wherein the oral administration of citric acid, or salts thereof, providing for drug enhancement and prolonging the paralytic effects of commercial neurotoxins for durations longer than commercial neurotoxin use without the enhancer.

13. The administration of citric acid, or salts thereof, as an active drug overwhelming the nicotinic receptors with acetylcholine through shunting enhancing the patient's ability to have a more stable mood or enhanced mood, increased pleasurable experiences, decreased pain including acute pain, chronic pain, neuropathic pain, nociceptive pain, somatic pain, visceral pain and can improve the cognition of those with diseases related to the nicotinic acetylcholine receptors.

14. The method of claim 13, wherein the administration of citric acid, or salts thereof, as an active drug overwhelming the nicotinic receptors with acetylcholine through shunting mitigating addiction cravings related to eating, smoking, opioids and all addictions related to acetylcholine or dopamine imbalances.

15. The method of claim 13, wherein the administration of citric acid, or salts thereof, as an active drug overwhelming the nicotinic receptors with acetylcholine through shunting causing an increase of dopamine to be used as a treatment for those with diseases with dopaminergic imbalance.

16. The method of claim 1, wherein the administration of citric acid, or salts thereof, causes calcium interference, local irritation and enhancement of aerobic respiration delaying the response to sprouting new nerve terminals and the formation of new synaptic contacts enhancing the longevity of commercial neurotoxin treatments.
